(19) United States
(12) Patent Application Publication
(10) Pub. No.: US 2021/0153865 A1
(43) Pub. Date: May 27, 2021

(54) Device and Method for Making Vertical Mattress Sutures with Surgical Staples

(71) Applicant: Muhammad Anwar, Madera, CA (US)

(72) Inventor: Muhammad Anwar, Madera, CA (US)

(21) Appl. No.: 17/045,649

(22) PCT Filed: Apr. 10, 2019
(86) PCT No.: PCT/US19/26817

§ 371 Date: Oct. 6, 2020

Related U.S. Application Data
(60) Provisional application No. 62/657,772, filed on Apr. 14, 2018.

(51) Int. Cl. A61B 17/068; A61B 17/076

(52) U.S. Cl. CPC A61B 17/0682; A61B 17/076

(57) ABSTRACT

The present invention is, in general, directed to a device for dispensing longer and shorter staples so that a user can close a wound or an incision with staples that close deeper tissue in the wound or incision and staples that close the superficial layers of skin in a cosmetically appealing manner.

### BACKGROUND OF THE INVENTION

Surgical staples (sometimes referred to as staple sutures) are used for the closing of many wounds or incisions in the tissues of a patient. In general, closing a wound or incision with surgical staples is a far easier procedure than conventional suturing. Of even greater importance, however, is the fact that the use of surgical staples is faster, thus reducing the time required for closing a wound or incision in a patient. This, in turn, reduces the length of time a patient must be maintained under anesthesia. Staples also tend to minimize wound inflammation, and are easier to remove than conventional sutures. Medical staples are removed with a staple remover tool, while conventional sutures are removed by cutting the suture next to the knot, then pulling the knot out gently until the suture is completely removed.

The vertical mattress suture is use to close skin wounds and has the advantage of providing closure for both deep and superficial layers of the skin. This suture technique allows perfect edge eversion, and vertical opposition of the superficial skin edges. The vertical mattress suture is most commonly used in anatomic locations which tend to invert, such as the posterior aspect of the neck, sites of greater skin laxity such as closure of lax skin, and for cosmetic purposes with areas of maximum exposure (e.g., the head and neck). It is useful for deep lacerations, where it can replace two layers of deep and superficial sutures. It can help bring the deep layers together alone, allowing several simple interrupted or running stitches to close the remaining superficial skin. The vertical mattress suture makes use of a deeper tissue sutures combined with sutures at the superficial layers of the wound or incision.

### SUMMARY OF THE INVENTION

In an aspect, the present invention provides a device for closing a subject's wound or incision with fastening members that close deep layers of tissue and other fastening members that close superficial layers of skin. Thus, the invention allows one to mimic the effect of deep and shallow sutures of the vertical mattress suture technique using fasteners such as surgical staples. The fastening members can be surgical staples made, for example, from titanium, stainless steel, or synthetic, bioabsorbable materials such as synthetic copolymers of polyglycolic acid:trimethylene carbonate (PGA:TMC) commercially sold as Gore® Seamguard®, or co-polymers of polylactide-polyglycolide commercially sold as the Insorb Absorbable Skin Staple or Polysorb® staples. In some embodiments, the staples are made of different lengths, for example, the longer staples may be 6-12 mm in length and 8-12 mm in width allowing closure of tissue at depths of 3-6 mm, and the shorter staples may be 3-6 mm in length and 3-6 mm in width allowing closure of superficial layers of tissue at depths of 1-3 mm.

In an aspect, the device is a stapler that has staples for closing deep layers of tissue, and staples for closing superficial layers of skin. In this aspect, the stapler contains longer staples for closing deeper layers of tissue (e.g., at depths of 3-6 mm) and the stapler contains shorter staples for closing superficial layers of skin (e.g., at depths of 1-3 mm). The staples may be organized in separate strips of staples, one strip for the longer staples and one strip for the shorter staples. Alternatively, the longer and shorter staples may be organized into a single strip of staples. In this embodiment, the longer and shorter staples may be arranged in an alternating order, or one or more staples of one length may be followed by one or more staples of the other length. In another alternative embodiment, one or more of one length of staple are followed by one or more of the other length of staple followed by one or more of the first length of staple, et cetera.

The stapler may contain two different hammers, one hammer for the longer staples and one hammer for the shorter staples. In the two hammer embodiment, each hammer may dispense and close staples at a different location on the stapler so that the stapler is repositioned to deliver the longer versus the shorter staples. Alternatively, each hammer may dispense and close staples at the same location on the stapler so that the stapler does not have to be repositioned to deliver the longer versus the shorter staples. In an alternative aspect, the stapler has one hammer which is adapted for both the longer and the shorter staples. In this embodiment, the stapler may dispense and close the shorter and longer staples at the same location on the stapler, or may dispense the shorter and longer staples at different positions on the stapler.

In an aspect, the present invention relates to methods of using the device of the invention for closing a subject's wound or incision. In this method of the invention, the same stapler is used to deliver the longer and shorter staples for closing a wound or an incision. In this method, the stapler uses the longer staples to close deep layers of tissue by puncturing deeper into the tissue to close the wound or incision at these deeper points. The stapler also uses the shorter staples to close the wound or incision at the superficial skin level by puncturing the superficial layers of the tissue surrounding the wound or incision with the shorter staples to bring together these upper layers of tissue (e.g., skin) in a cosmetically appealing manner. In some embodiments, the longer staples are applied first to close the deep layers of the wound or incision, followed by application of the short staples to close the upper layers of tissue (e.g., skin) in a cosmetically appealing manner. In an alternate embodiment, the longer and shorter staples are applied in an alternating format where one or more long staples are applied, followed by one or more short staples, followed by one or more long staples, etc. The first staple(s) applied in this alternating embodiment may be the longer or shorter staples.

In an aspect, the present invention also relates to methods using fastening members to close deep layers of tissue and fastening members for closing superficial layers of skin. In these methods of the invention, longer fasteners are used to close deep layers of tissue where the longer fasteners puncture deeper into the tissue and close the wound or incision at these deeper points. Shorter fasteners are used to close the wound or incision at the superficial skin level using shorter fasteners that puncture the superficial layers of the tissue surrounding the wound or incision and bring together these upper layers of tissue (e.g., skin) in a cosmetically appealing manner.

### BRIEF DESCRIPTION OF THE FIGURES

FIG. 1 shows a depiction of an embodiment for a surgical stapler remover.

FIG. 2 shows an overhead view of one embodiment of the shorter staples (microstaples) and longer staples (regular staples) of the invention.

FIG. 3 shows a side view of a device with shorter staples (microstaples) and the longer staples (regular staples).

FIG. 4 shows an end view of device with two hammers, one for the shorter staples (microstaples) and one for the longer staples (regular staples).

FIG. 5 shows the hammer acting on a staple to close the staple.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

Reference throughout the specification to “various embodiments,” some embodiments,” one embodiment,” or an embodiment“, or the like, means that a particular feature, structure, or characteristic described in connection with the embodiment is included in at least one embodiment. Thus, appearances of the phrases in various embodiments,” in some embodiments,” in one embodiment“, or in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation. Such modifications and variations are intended to be included within the scope of the present invention.

The terms “proximal” and “distal” are used herein with reference to a user manipulating the handle portion of the device. The term “proximal” referring to the portion closest to the user and the term “distal” referring to the portion located away from the user. It will be further appreciated that, for convenience and clarity, spatial terms such as “vertical”, “horizontal”, “up”, and “down” may be used herein with respect to the drawings. However, devices of the invention are used in many orientations and positions, and these terms are not intended to be limiting and/or absolute.

Various exemplary devices and methods are provided for closing wounds and incisions, and performing minimally invasive procedures. However, the person of ordinary skill in the art will readily appreciate that the various methods and devices disclosed herein can be used in numerous procedures and applications including, for example, in connection with open surgical procedures. As the present Detailed Description proceeds, those of ordinary skill in the art will further appreciate that the various instruments disclosed herein can be applied to the surface or inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongated shaft of a surgical instrument can be advanced. Thus, the present invention can be utilized in a variety of conditions. The configuration of a device of the invention may depend on the use for which the device is intended and the conditions under which the device will operate.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 it is intended that the concentration be understood to be at least approximately or about 10 μg.

As used herein, the term “cosmetically appealing” is defined to mean that a scar appears more consistent with the skin tone of the tissue around the scar. For example, a scar that is thin, flat, and closely matches the skin tone of adjoining skin is cosmetically appealing. Proper alignment of skin on the edges of the wound of incision also leads to a cosmetically appealing scar.

As used herein, the term “longer staples” is defined to mean staples that close tissue at depths of 3-8 mm.

As used herein, the term “shorter staples” is defined to mean staples that close tissue at 1-3 mm in depth.

### Stapling Device

In an aspect, the stapler device of the invention can be any surgical stapler device known in the art wherein the known stapler device has been adapted to deliver longer staples (regular staples) for closing deeper layers of tissue and shorter staples (microstaples) for closing superficial layers of tissue in a cosmetically pleasing way. Surgical stapler devices that can be modified to devices of the invention include surgical staplers that are commercially sold, for example, the Appose Single Use Skin Stapler, 8886803512, DFS Single Use Fascia Stapler, 070614, MultiFire Premium Single Use Skin Stapler, 059137, 059035, 059037, the Signet Single Use Skin Stapler, 054006, and the SFS Reusable Skin Stapler, 060210, all sold by Covidien Medtronic, 3M Precise Multishot Skin Stapler, 3M Precise Vista Skin Stapler, 3M Precise PGX Disposable Skin Stapler, MedCare PR Reflex One Skin Stapler, Ethicon Proximate PX 35W Fixed Head Skin Stapler, and Conmed Reflex One Skin Stapler.

In an aspect, the device of the invention is a surgical stapler. In some embodiments, the surgical stapler is a hand device with a handle part which is gripped in the user's palm and a pivotal or otherwise movable trigger part which is moved when squeezed by the user's fingers against the adjacent handle. The trigger is an elongated element with a pivot point close to the front end so that movement of one end causes opposite motion of the remote end. Within the stapler the trigger, driven by the user's hand, is the basic power source which is transmitted through various components to deliver and close each staple with a hammer.

The staples may be organized in separate strips of staples, one strip for the longer staples and one strip for the shorter staples. Alternatively, the longer and shorter staples may be organized into a single strip of staples. In this embodiment, the longer and shorter staples may be arranged in an alternating order, or two or more staples of one length may be followed by one or more staples of the other length. In another alternative embodiment, one or more of one length of staple are followed by one or more of the other length of staple followed by one or more of the first length of staple, et cetera.

The stapler may contain two different hammers, one hammer for the longer staples and one hammer for the shorter staples. In the two hammer embodiment, each hammer may dispense and close staples at a different location on the stapler so that the stapler is repositioned to deliver the longer versus the shorter staples. Alternatively, each hammer may dispense and close staples at the same location on the stapler so that the stapler does not have to be repositioned to deliver the longer versus the shorter staples. In an alternate embodiment, the stapler has one hammer which is adapted for both the longer and the shorter staples. In this embodiment, the stapler may dispense and close the shorter and longer staples at the same location on the stapler, or may dispense the shorter and longer staples at different positions on the stapler.

### Fastening Members

The fastening members can be any fastening member that can be used to close a wound or incision. In an aspect, the fastening members of the invention are surgical staples made, for example, from titanium, stainless steel, or synthetic, bioabsorbable materials such as synthetic copolymers of polyglycolic acid:trimethylene carbonate (PGA:TMC) commercially sold as Gore® Seamguard®, or co-polymers of polylactide-polyglycolide commercially sold as the Insorb Absorbable Skin Staple or Polysorb® staples.

Exemplary surgical staples include those sold commercially, including for example, the staples made by Novo Precision, Insorb absorbable skin staples sold by Incisive Surgical, Inc., or any of the staples used in the surgical staplers listed above.

In some embodiments, the longer staples (regular staples) are typically 6-12 mm in length by 8-12 mm in width, including, for example, 6 mm by 8 mm, 6 mm by 10 mm, 6 mm by 12 mm, 8 mm by 8 mm, 8 mm by 10 mm, 8 mm by 12 mm, 10 mm by 8 mm, 10 mm by 10 mm and 10 mm by 12 mm, 12 mm by 8 mm, 12 mm by 10 mm, and 12 mm by 12 mm. In some embodiments, the shorter staples (microstaples) are typically 3-6 mm in length and 3-6 mm in width, including, for example, 3 mm by 3 mm, 3 mm by 4 mm, 3 mm by 5 mm, 3 mm by 6 mm, 4 mm by 3 mm, 4 mm by 4 mm, 4 mm by 5 mm, 4 mm by 6 mm, 5 mm by 3 mm, 5 mm by 4 mm, 5 mm by 5 mm, 5 mm by 6 mm, 6 mm by 3 mm, 6 mm by 4 mm, 6 mm by 5 mm, and 6 mm by 6 mm.

### A Staple Remover for the Shorter Staples

Removal of the shorter staples can be best accomplished with modified staple removers. Standard staple removers may be too wide for the shorter staples and may cause damage to the skin and/or wound/incision if used to extract the shorter staples. In some embodiments, the staple remover is adapted for removing just the shorter staples. In some embodiments, the staple remover is adapted for removing both the shorter and the longer staples. Staple removers that may adapted for removal of the shorter staples include those which are known in the art and/or are sold commercially. For example, the staple removers that may be so modified are disclosed in U.S. Pat. Nos. 8,690,896, 8,241,303, 8,177,793, 5,916,223, 5,658,297, 5,451,231, 4,805,876, and 4,515,348, and Design Pat. Nos. D0438,965, D0407,489, D0302,466, D0287,279, D0283,048, and D0281,624. Other surgical staple removers that may be modified include, for example, skin staple removers sold by Ethicon PSX, FE.MA SU11.0012, 3M Precise SR-3 and Precise SR-1, Dynarex SKU 4522, and Medline model nos. 716-IMC, DYNJ04058.

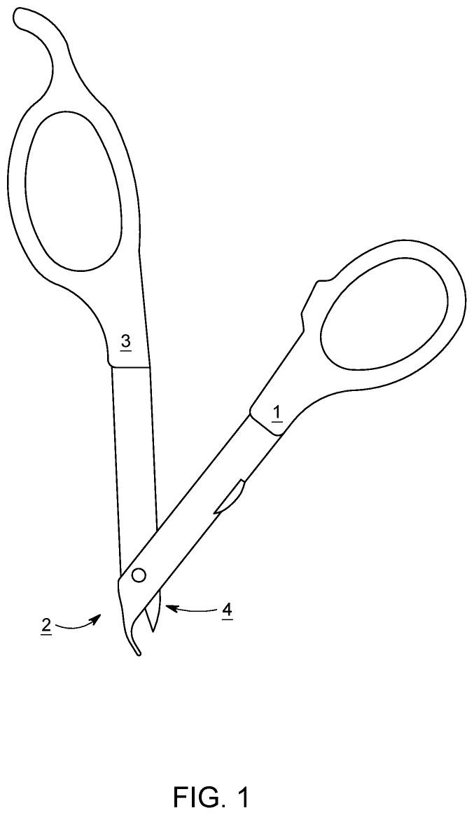

In some embodiments, a staple remover for use with longer (or standard) staples is adapted to remove the shorter staples by modifying the anvils of the surgical staple remover to be thinner and longer so it is compatible with the shorter staples. In some embodiments, the staple remover is a manual remover such as that depicted in FIG. 1. This embodiment is a scissors type remover. The handle element (1) is bent from flat to V-shaped and the end (2) is an anvil that slides under the staples during the extraction process. The handle element (3) ends in a staple extraction blade (4) that together with the anvils removes staples from a subject. Both the anvil (2) and the staple extraction blade (4) are adapted to the smaller size of the shorter staples so that the staple remover can act on the shorter staples that are placed in the superficial layers of tissue from the subject. The anvil (2) may be forked with one stem on either side of the staple extraction blade, or the anvil maybe solid with raised areas on either side of the staple extraction blade. In some embodiments, the anvil is adapted to receive both the shorter staples and the longer staples, and so the staple remover can remove both the longer staples (deeper tissue penetration) and the shorter staples (superficial tissues) from the subject.

### Methods of Using the Longer and Shorter Staples

The vertical mattress suture is made with a deep loop at about 4-8 mm in depth followed by a shallow loop at about 1-2 mm in depth. In an embodiment of the invention, staples are used to mimic the vertical mattress suture by applying longer staples that will close a wound or incision at about 4-8 mm in depth with shorter staples that close a wound or incision at 1-2 mm in depth. In an alternative embodiment, the longer staples can close a wound or incision at 3-6 mm in depth and the shorter staples can close a wound of incision at 1-3 mm.

In some embodiments, the longer staples are applied to bring together deeper layers of tissue in the wound or incision, and the shorter staples are applied to bring together superficial layers of skin in a cosmetically appealing manner. The longer staples may be applied first to bring together the deeper layers of tissue in a wound or incision, followed by application of the shorter staples to align the edges of the wound or incision in a cosmetically appealing manner. The longer staples and shorter staples may be applied in an alternating fashion, one or more long (or short) staples may be applied first followed by one or more shorter staples followed by one or more longer staples, etc., or many longer staples (or shorter staples) may be applied first followed by application of many shorter (or longer) staples.

In some embodiments, the staples may have three or more different lengths depending on the application to which the staples are used. As with the two lengths of staples embodiment, the embodiments with three or more lengths of staples can apply the staples of different lengths in any order and any alternating pattern at the discretion of the user.

The methods of applying the longer and shorter staples of the invention may be achieved using the stapler devices of the invention which dispenses both the longer and shorter staples. In this aspect of the invention, the same stapler is used to deliver the longer and shorter staples for closing a wound or an incision. In this method, the stapler uses the longer staples to close deep layers of tissue by puncturing deeper into the tissue to close the wound or incision at these deeper points. The stapler also uses the shorter staples to close the wound or incision at the superficial skin level by puncturing the superficial layers of the tissue surrounding the wound or incision with the shorter staples to bring together these upper layers of tissue (e.g., skin) in a cosmetically appealing manner. In some embodiments, the longer staples are applied first to close the deep layers of the wound or incision, followed by application of the shorter staples to close the upper layers of tissue (e.g., skin) in a cosmetically appealing manner. In an alternate embodiment, the longer and shorter staples are applied in an alternating format where one or more longer staples are applied, followed by one or more shorter staples, followed by one or more longer staples, etc. The first staple(s) applied in this alternating embodiment may be the longer or shorter staples.

Alternatively, two or more staplers may be used to apply the two or more staples of different length. In this alternative aspect, two devices are used and one device has staples for closing deep layers of tissue and the other device has staples for closing superficial layers of skin. In this method, a first device is used to close deep layers of tissue using longer staples that puncture deeper into the tissue and close the wound or incision at these deeper points. The second device is used to close the wound or incision at the superficial skin level using shorter staples that puncture the superficial layers of the tissue surrounding the wound or incision and bring together these upper layers of tissue (e.g., skin) in a cosmetically appealing manner. In some embodiments, the longer staples are applied first to close the deep layers of the wound or incision, followed by application of the short staples to close the upper layers of tissue (e.g., skin) in a cosmetically appealing manner. In an alternate embodiment, the longer and shorter staples are applied in an alternating format where one or more long staples are applied, followed by one or more short staples, followed by one or more long staples, etc. The first staple(s) applied in this alternating embodiment may be the longer or shorter staples.

The inventions disclosed herein will be better understood from the examples which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: A Stapling Apparatus for Delivering Longer and Shorter Staples

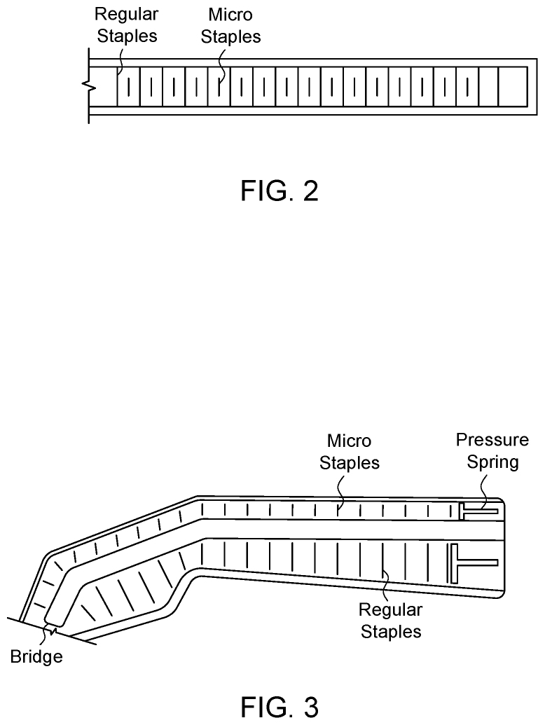

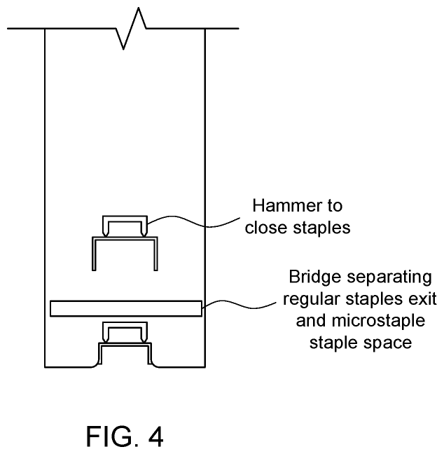

FIG. 2 depicts a top view of one aspect of a stapler embodiment of the invention. In this view, the longer (regular) staples and smaller (micro) staples are seen as two rows of staples with the row of smaller (micro) staples being above the row of longer (regular) staples. FIG. 3 is a side of view of this same embodiment of a stapler of the invention. In this side view, the smaller (micro) staples are above the longer (regular) staples and each row of staples is urged forward by a pressure spring. In this embodiment, the smaller (micro) staples and the longer (regular) staples are dispensed from separate openings in the stapler. FIG. 4 shows an end view of this stapler embodiment with two openings, one for each hammer that dispenses and closes the staples at its respective opening. In this embodiment, the longer (regular) staples are dispensed from an opening with a hammer that dispenses and closes the longer staples, and the shorter (micro) staplers are dispensed and closed from the other opening with a hammer the dispenses and closes the shorter staples.

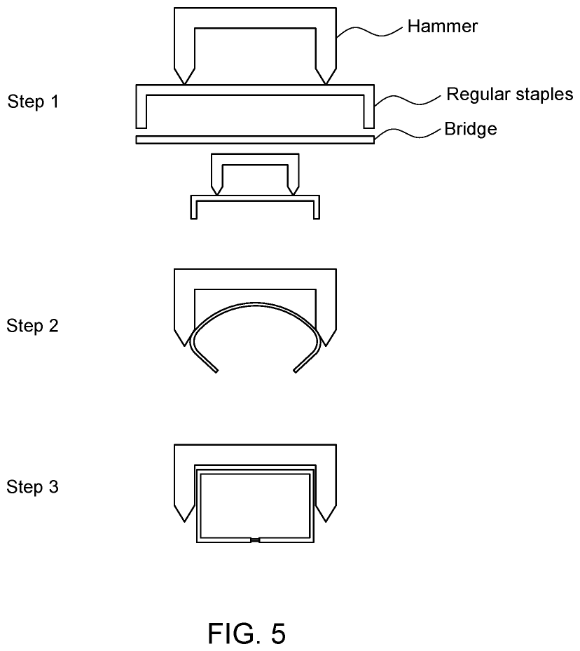

FIG. 5 shows a one hammer embodiment for closing the longer and shorter staples of the invention. In this embodiment, the hammer presses down on an individual staple, separates the staple from the row (step 1), and pushes the staple through tissue as the staple (step 2) is bent into the closed position (step 3).

The stapler of some embodiments may be designed with separate trigger mechanisms for actuating each of the hammers (in the two hammer embodiment), or the staple may have a single trigger mechanism that can be switched from one hammer of the other to deliver the longer or shorter staples as desired. In the one hammer embodiment of the invention, the stapler can have one trigger mechanism for actuating the single hammer to dispense and close either the shorter or the longer staples.

### Example 2: Using a Stapling Device to Mimic Vertical Mattress Sutures

The device of Example 1 may be used to close a wound or incision in a manner mimicking a vertical mattress suture. The stapler of Example 1 is used to apply the longer and shorter staples to the wound or incision. In an embodiment, the stapler first applies longer staple(s) to bring together the deeper tissue of the wound or incision. Then the stapler applies shorter staple(s) to bring together the superficial layers of tissue (e.g., skin) in a cosmetically appealing fashion. For example, the skin layers can be joined together so that the skin at the edges of the wound are aligned to minimize scar formation.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method for closing a wound or an incision, comprising the steps of: applying longer staples to close a deeper layer of tissue in the wound or the incision, and applying shorter staples to close superficial layers of tissue in the wound or the incision.

2. The method of claim 1, wherein the shorter staples close the superficial layers of tissue in the wound or the incision so as to minimize scar formation.

3. The method of claim 1, wherein the shorter staples close the superficial layers of tissue in the wound or the incision so that the edges of the wound or incision are aligned.

4. The method of claim 1, wherein the shorter staples close the superficial layers of tissue in the wound or the incision in a cosmetically appealing manner.

5. The method of claim 1, wherein the longer staples are 6 mm to 12 mm in length, and the width of the staples is 8 mm to 12 mm.

6. The method of claim 5, wherein the longer staples are 6, 8, 10 or 12 mm in length.

7. The method of claim 6, wherein the longer staples are 8 mm in length.

8. The method of claim 5, wherein the longer staples are 8, 10, or 12 mm in width.

9. The method of claim 6, wherein the longer staples are 8 mm in width.

10. The method of claim 1, wherein the shorter staples are 3 mm to 6 mm in length, and the width of the staples is 3 mm to 6 mm.

11. The method of claim 5, wherein the shorter staples are 3, 4, 5, or 6 mm in length.

12. The method of claim 6, wherein the shorter staples are 4 mm in length.

13. The method of claim 5, wherein the shorter staples are 3, 4, 5, or 6 mm in width.

14. The method of claim 6, wherein the shorter staples are 4 mm in width.

15. (canceled)

16. (canceled)

17. (canceled)

18. (canceled)

19. (canceled)

20. (canceled)

21. (canceled)

22. (canceled)

23. (canceled)

24. (canceled)

25. (canceled)
